(19) United States
(12) Patent Application Publication
(10) Pub. No.: US 2020/0197722 A1
(43) Pub. Date: Jun. 25, 2020

(54) Method for Adjusting Level of Galanin in Brain by Optic Nerve Light Conduction

(71) Applicant: Tong Li, Beijing (CN)

(72) Inventors: Tong Li, Beijing (CN); Xiaogang Wang, Richmond Hill (CA)

(73) Assignee: Tong Li

(21) Appl. No.: 16/808,557

(22) Filed: Mar. 4, 2020

(51) Int. Cl. A61N 5/06

(52) U.S. Cl. CPC A61N 5/0622; A61N 2005/0629; A61N 2005/0663; A61N 2005/0654; A61N 2005/0657; A61N 2005/0658; A61N 2005/0652; A61N 2005/0667

(57) ABSTRACT

The present invention discloses a method for adjusting the level of galanin in brain, by optic nerve light conduction, comprising the following step: controlling light of different wavelengths, different intensities and different frequencies to illuminate an eye by a light adjustment device, so that galanin receptors in the eye are activated to produce signal, stimulation, and signals transmitted to the hypothalamic visual receiving area via the optic nerve can influence the level of galanin expression in the brain.

### TECHNICAL FIELD

The present invention relates to a method for adjusting the level of galanin in brain by optic nerve light conduction.

### BACKGROUND

Galanin (GAL) is a regulatory peptide consisting of 30/29 (human/rodent) amino acids. GAL is broadly distributed in the central and peripheral nervous systems as well as in non-neuronal tissues. Galanin peptide has a wide range of nonneuronal functions as well as classic neuro-modulatory roles. Galanin is important for a variety of biological functions such as feeding, pain processing, endocrine functions, neuroprotection, sleep, mood change, and sexual reproduction. It is believed that galanin expression level might be especially closely related to the pathogenesis of Alzheimer's disease (AD), anxiety and depression, insomnia, and sensitivity of pain caused by nerve injury.

Galanin has been shown to exerts its effects on numerous biological and pathophysiological processes through three G-protein coupled receptors GLAR 1-3. In the cornea, GALR1-3 are detected in the stratum basal of epithelial cells, stromal cells and endothelial cells, and adjacent conjunctiva (Distribution of galanin receptors in the human eye, (Falk Schrodl, et al, Experimental Eye Research 138 (2015) 42-51). In the iris, GALR1-3 are found in the sphincter and dilator, and also in iris vessels. In the ciliary body, GALR1 are found only in non-pigmented epithelial cells, while GALR3 are found in the ciliary muscles and vessels. In the retina, GALRrs are present in a large number of cells and GLAR 2's in a small number of cells of GALR2; GALR3's are also present in a small number of neurons and GALR2's are also found around the retinal vessels and retinal pigment epithelial cells together with GALR3. In the choroid, the presence of GALR1-3 are detected in both endogenous choroidal neurons and nerve fibers of the choroidal stroma, and these three receptors may also be detected around the choroidal vessels, while the choroidal vascular layer seems to be only containing GALR3. Also GALR 1-3 present in the eye might be related to the wound healing or inflammation process, vascular dynamics and signal transduction.

Since the human eyes convert visual input into electrical information and route this information to the brain, research has shown that light of different wavelengths and frequencies (including visible light and invisible light) could generate different signal stimulation for GALR.

Further, research shows that light of different wavelengths and frequencies (including visible light and invisible light) may generate different signal stimulations for GALR. A marked alternation in galanin expression in the brain is observed under a number of conditions. This suggests a role for the neuropeptide and tis receptors for a novel therapeutic method. To be more specific, adjusting GAL in the brain by using GALR in the eye could influence the galanin expression in the brain. Due to the critical role of galanin in many biological processes, this method could have a wide range of applications.

### SUMMARY

In view of this, the present invention provides a method for adjusting the level of galanin in the brain by optic nerve light conduction. The key idea or procedure is to use light of different wavelengths, different intensities and different frequencies to illuminate an eye by a light adjustment device in order to activate the galanin receptors in the eye to generate signals transmitted to the hypothalamic via the optic nerve. The light of different wavelengths, different intensities and different frequencies include visible light and invisible light.

The process of controlling the light of different wavelengths, different intensities and different frequencies to illuminate an eye will be done through filtering natural light or simulated natural light according to wavelengths in the, order form large to small and light intensities and from strong to weak, until all light is blocked or turned off

The process of controlling the light of different wavelengths, different intensities and different frequencies to illuminate an eye also comprises gradually recovering the light source according to wavelengths in the order from small to large and light intensities from weak to strong in the absence of other light source, until the natural light or the simulated natural light gradually illuminates the human eye.

The wavelengths are in the order from large to small as follows: red light: 605-720 nm, orange light: 595-605 nm, yellow light: 580-595 nm, yellow-green light: 560-580 nm, green light: 500-560 nm, blue-green light: 490-500 nm, cyan light: 480-490 nm, blue light: 450-480 nm, and purple light: 400- 435 nm.

The light adjustment device comprises a light, source, light filters and a light filter adjustment assembly; wherein the light source is, natural light or simulated natural light using light-emitting devices; light filters which are used to filter light of different wavelengths respectively; and the light filter adjustment assembly is used for adjusting the position of the multiple light filters in order for the light filters to be located either between the light source and the eye or be removed completely.

The light adjustment device can be configured into an eyeshade-type product, and a conventional mobile or wearable component may be also selected as the light filter adjustment device.

In summary, the light adjustment apparatus comprises a light source, a light wavelength control unit and a light frequency control unit, wherein the light source of the device is an array of light-emitting elements of different wavelengths; the light wavelength control unit controls the light-emitting elements of different wavelengths to emit light, respectively through current connection; and the light frequency control unit controls the light-emitting intensities of the light-emitting elements of different wavelengths respectively through current pulse width. In the present invention, by applying repeated and well controlled light stimulation on the eye through light source signals of different wavelengths and frequencies, GALR in the eye may be activated, thus achieving the purpose of adjusting the level of the GAL in brain through optic nerve conduction.

### DETAILED DESCRIPTION

### Embodiment 1

A method for adjusting the, level of galanin in brain by optic nerve light conduction, comprising the following, step: controlling light of different wavelengths and different frequencies to illuminate an eye by a light adjustment device, so that galanin receptors in the eye are activated to produce signal stimulation, and signals transmitted to the hypothalamic visual receiving area via the optic nerve can then influence the level of galanin expression in the brain.

The light adjustment device comprises a light source, multiple light filters and a light filter adjustment assembly, wherein the light source is simulated natural, light using light-emitting elements; the light filters are multiple in number, which are used, for filtering light of different wavelengths, respectively; and the light filters are mounted on the light filter adjustment assembly; and the light filter adjustment assembly is used for adjusting the position of the multiple light filters to enable the light filters to be located between the light source and the eye or to be removed when necessary.

The light adjustment device is configured into an eyeshade-type product and is worn on the head while in use. The simulated natural light emitted by the light-emitting elements is made to illuminate the eye; then the light filter adjustment assembly is controlled to gradually superimpose the light filters between the light source and the eye according to wavelengths in the order from large to small to filter light, until all light are blocked or turned off.

The wavelengths are in the order from, large to small as follows: red light: 605-720 nm, orange light: 595-605 nm, yellow light: 580-595 nm, yellow-green light: 560-580 nm, green light: 500-560 nm, blue-green light: 490-500 nm, cyan light: 480-490 nm, blue light: 450-480 nm, and purple light: 400- 435 nm.

Further, the light filters may be gradually removed according to wavelengths in the order from small to large, until the simulated natural light gradually illuminates the human eye.

### Embodiment 2

A method for adjusting the level of galanin in brain by optic nerve light conduction, comprising the following step: controlling light of different wavelengths and different frequencies to illuminate an eye by a light adjustment device, so that galanin receptors in the eye are made to produce signal stimulation, and signals transmitted to the hypothalamic visual receiving area via the optic nerve to influence the level of galanin in the brain.

The light adjustment device comprises a light source, a light wavelength control unit and a light frequency control unit, wherein the light source is an array of light-emitting elements of different wavelengths; LED lamps of different light wavelengths may be arranged to form the array; the light wavelength control unit may select, any form of current control elements to control currents of light-emitting elements of different wavelengths respectively, so that light-emitting elements of different wavelengths are turned on or turned off, and then illumination of light of different wavelengths is achieved; and the light frequency control unit may be set to control and adjust, the light-emitting intensities of the light-emitting elements of different wavelengths respectively.

The light adjustment device is configured into an eyeshade-type product (encapsulated in the eyeshade shell) and is worn on the outside of the eye while in use; all light-emitting elements in the array are controlled to emit light through current, the simulated natural light is made to illuminate the eye, then the light frequency control unit is controlled to reduce the light-emitting intensity of the light-emitting element of the maximum wavelength, the light-emitting element is controlled to be turned off by the light wavelength control unit, and the above-mentioned operation is repeatedly performed on the light-emitting elements of different wavelengths according to wavelengths in the order from large to small, until all light are turned off.

The wavelengths are in the order from large to small as follows: red light: 605-720 nm, orange light: 595-605 nm, yellow light: 580-595 nm, yellow-green light: 560-580 nm, green light: 500-560 nm, blue-green light: 490-500 nm, cyan light: 480-490 nm, blue light: 450-480 nm, and purple light: 400-435 nm.

Further, the light-emitting elements may be gradually turned on according to wavelengths in the order from small to large and light intensities in the order, from weak to strong, until the simulated natural light gradually illuminates the human eye.

By adjustment of light of different wavelengths and different frequencies, GALR 1-3 in the eye may be activated to achieve the purpose of adjusting the level of the GAL in brain through optic nerve conduction. Since GAL is involved in mediating a variety of biological functions such as feeding, sense of pain, endocrine, neuroprotection, learning and memory, sleep, mood, sexuality and reproduction, it is expected to realize the intervention of biological response in vivo by adjustment of light of different wavelengths and different frequencies as a novel therapeutic measure.

### Embodiment 3

A method for adjusting the level of galanin in brain by optic nerve light conduction, comprising the following step: making blue light of 8-10 Hz and red light of 0.5-4 Hz illuminate an eye by a light adjustment device in order to activate the GLAR 1-3 in the eye to achieve signal stimulation; and signals transmitted to the hypothalamic visual receiving area via the optic nerve can influence the level of galanin in the brain.

The light adjustment device comprises a light source, a light wavelength control unit and a light frequency control unit, wherein the light source is an array formed by arranging red light LED lamps and blue light LED lamps; the light wavelength control unit may select any form of current control elements to control currents of the red light LED lamps and the blue light LED lamps respectively, so that the red light LED lamps or the blue light LED lamps are turned on or turned off; and the light frequency control unit may select any form of current pulse width, control elements to control the light-emitting intensities of the red light LED lamps and the blue light LED lamps respectively, thus adjusting the intensities of light.

The light adjustment device is, configured into an eyeshade-type product and is worn on the eye while in use, the red light LED lamps in the array are, controlled to emit light through the current, and then the light frequency control unit is controlled to adjust the frequency to 0.5-4 Hz, thus reducing the level of galanin in the brain and promoting sleep. The blue light LED lamps in the array are controlled to emit light through the current, and then the light frequency control unit is controlled to adjust the frequency to 8-10 Hz, thus increasing the level of galanin in the brain and promoting wakeup.

The above description of the disclosed embodiments enables those skilled to realize or use the present invention. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

1. A method for adjusting the level of galanin in brain by optic nerve light conduction, comprising the following step: controlling light of different wavelengths, different intensities and different frequencies to illuminate an eye by a light adjustment device, so that galanin receptors in the eye are activated to produce signal stimulation, and signals transmitted to the hypothalamic visual receiving area via the optic nerve can influence the level of galanin in the brain.

2. The method for adjusting the level of galanin in brain by optic nerve conduction according to claim 1, wherein the light of different wavelengths, different intensities and different frequencies include visible light and invisible light.

3. The method for adjusting the level of galanin in brain by optic nerve light conduction according to claim 1, wherein the process of controlling the light of different wavelengths, different intensities and different frequencies to illuminate an eye comprises: gradually filtering natural light or simulated natural light according to wavelengths in the order from large to small and light intensities in the order from strong to weak, until all light are blocked or turned off.

4. The method for adjusting the level of galanin in brain by optic nerve light conduction according to claim 3, wherein the wavelengths are in the order from large to small as follows: red light: 605-720 nm, orange light: 595-605 nm, yellow light: 580-595 nm, yellow-green light: 560-580 nm, green light: 500-560 nm, blue-green light: 490-500 nm, cyan light: 480-490 nm, blue light: 450-480 nm, and purple light: 400- 435 nm.

5. The method for adjusting the level of galanin in brain by optic nerve light conduction according to claim 1, wherein the process of controlling the light of different wavelengths, different, intensities and different frequencies to illuminate an eye comprises: gradually recovering the light source according to wavelengths in the order from small to large and light intensities in the order from weak to strong in the absence of light source, until the natural light or the simulated natural light gradually illuminates the human eye.

6. The method for adjusting the level of galanin in brain by optic nerve light conduction according to claim 1, wherein the light adjustment device comprises a light source, light filters and a light, filter adjustment assembly, wherein the light source is natural light or simulated natural light simulated using light-emitting elements; the light filters are multiple in number, which are used for filtering light of different wavelengths respectively; and the light filter adjustment assembly is used for adjusting the position of the multiple light filters, to enable the light filters to be located between the light source and the eye or be removed between the light source and the eye.

7. The method for adjusting the level of galanin in brain by optic nerve light conduction, according to claim 1, wherein the light adjustment device comprises a light source, a light wavelength control unit and a light frequency control unit, wherein the light source is an array of light-emitting elements of different wavelengths; the light wavelength, control unit controls the light-emitting elements of different wavelengths to emit light respectively through current connection; and the light frequency control unit controls the light-emitting intensities of the light-emitting elements of different wavelengths respectively through current pulse width control elements.
